(19) United States
(12) Patent Application Publication
(10) Pub. No.: US 2006/0106099 A1
(43) Pub. Date: May 18, 2006

(54) Novel inhibitors of ubiquitin isopeptidases

(76) Inventor: James E Mullally, (US)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/521,570

(22) PCT Filed: Jul. 18, 2003
(86) PCT No.: PCT/US03/22576

§ 371 Date: Nov. 7, 2005

(30) Foreign Application Priority Data
Jul. 18, 2002 (US) 60395584

(51) Int. Cl. A61K 31/557; A61K 31/366; A61K 31/336; A61K 31/12

(52) U.S. Cl. 514/475; 514/573; 514/690

(57) ABSTRACT

A novel class of inhibitors of ubiquitin isopeptidases is disclosed that cause tumor cell death via molecular mechanisms independent of p53. Specifically, compounds containing an a,β-unsaturated ketone with a sterically accessible electrophilic β-carbon and related compounds are identified herein. Pharmaceutical compositions comprising the inhibitor compounds and methods of using the compounds for treating a variety of disease states are disclosed.

### BACKGROUND OF THE INVENTION

1. Field of the Invention

The invention provides a class of inhibitors of ubiquitin isopeptidases. The invention also provides to pharmaceutical compositions and therapeutic methods of using the same.

2. Description of the Background of the Invention

The tumor suppressor p53 is mutated in greater than fifty percent of all cancers. Importantly, most clinically useful antineoplastic agents are less potent and efficacious in the context of mutant p53. This situation has prompted a search for agents that cause tumor cell death via molecular mechanisms independent of p53.

Ubiquitin isopeptidases, i.e., ubiquitin specific proteases, are a family of cysteine proteases that salvage ubiquitin for reuse by the 26S proteasome system (Hochstrasser, 1996; Hershko and Ciechanover, 1998) and regulate the activity of a variety of substrates by altering heir ubiquitination status. The ubiquitin salvage activity of the isopeptidases cleaves the isopeptide bond between the C-terminal carboxyl of ubiquitin and the ε-amino group of a lysine residue on an adjacent protein This disassembles ubiquitin oligomers, ubiquitin-protein conjugates, and ubiquitin-peptide conjugates and maintains a cellular pool of monomeric ubiquitin by.

Few inhibitors of isopeptidases have been identified, other than analogs based on ubiquitin itself. These include non-hydrolyzable ubiquitin dimer analogs (˜16 kDa) (Yin et al., 2001) and ubiquitin aldehyde (˜8.5 kDa) (Dang et al., 1998); molecules which primarily are suitable for investigating isolated enzymes. Such molecules are not cell permeable, are not active in intact cells, and are not orally active.

Mullally et al. (2001) identified Δ12-prostaglandin J₂ (Δ2-PGJ₂) as a novel isopeptidase inhibitor that is active in intact cells but found that other prostaglandins were less active, or inactive. Thus, PGA₁, PGA₂, and 15-keto-PGs were significantly less potent Δ2-PGJ₂. PGB₁ was inactive . It was posited that J series prostaglandins containing a cross-conjugated dienone structure would be more efficacious than single 1,2 unsaturated ketone compounds, but there was no teaching or suggestion that compounds other than unsaturated prostaglandins and prostaglandin analogs would be active as isopeptidase inhibitors.

It is apparent, therefore, that new and potent isopeptidase inhibitors, and compositions comprising isopeptidase inhibitors which inhibit tumor growth are greatly to be desired. It is also apparent that methods of treating cancer using such inhibitors is highly desirable.

### SUMMARY OF THE INVENTION

Accordingly, the invention provides a new class of compounds which cause tumor cell death via molecular mechanisms independent of p53. Namely, compounds having an α,β-unsaturated ketone with a sterically accessible electrophilic β-carbon are identified herein as a class of ubiquitin isopeptidases inhibitors, and, furthermore, inhibitors of ubiquitin isopeptidases cause cell death in vitro independently of p53. The present invention does not include cyclopentenone prostaglandins of the J family.

In accordance with a first embodiment of the invention there is provided a method of inhibiting one or more ubiquitin isopeptidases in a cell, by contacting the cell with an effective amount of a composition comprising a compound having an α,β-unsaturated ketone, where the ketone has a sterically accessible electrophilic β-carbon, and where the agent is cell permeable and active in intact cells, and where the agent is not a cyclopentenone prostaglandin of the J family. The cell may be a human cell.

In accordance with a second aspect of the invention there is provided a method of treating or alleviating an oncological malady in a subject, comprising administering to the subject a composition comprising an effective amount of a ubiquitin isopeptidase inhibitor. The subject may be a human subject, and the oncological malady may be, for example, a malady selected from the group consisting of tumors of the head and neck, esophagus, stomach, ileum, colon, rectum, breast, ovary, prostate, testes, lung, brain, kidney, liver, pancrease, muscle (sarcoma), connective tissue (sarcoma) or fat (sarcoma),bone marrow, lymphomas and leukemias.

In these methods, the compound may contain a cross-conjugated α,β, α′,β′-unsaturated ketone moiety, where one or both of the electrophilic β carbons is sterically accessible. Moreover, the α carbon of at least one α,β-unsaturated ketone moiety may bear an electron withdrawing substituent. The electron withdrawing substituent may be, for example, a fluorine, chlorine, bromine, iodine, nitro, nitrilo or carboxy group. The carboxy group may be, for example, an acid, ester of amide group.

In a particular embodiment, the α,β-unsaturated ketone may comprise a conjugated cyclopentene moiety. In particular embodiments, the ketone may be selected from the group consisting of dibenzylideneacetone (DBA), curcumin, shikoccin (NSC-302979), shikoccin epoxide, O-methyl shikoccin, O-methyl shikoccin epoxide, shikodomedin, rabdoshikoccin A, rabdoshikoccin B, rabdolatifolin, rabdoumbrasanin, and a punaglandin. The punaglandin may be selected, for example, from the group consisting of PNG 2, PNG3, PNG4, Z-PNG-4, and PNG 6.

In accordance with another aspect of the invention there is provided a pharmaceutical composition suitable for treating an oncological malady in a human subject, comprising an effective amount of a ubiquitin isopeptidase inhibitor, where the inhibitor is not a cyclopentenone prostaglandin of the J family. The composition may comprise an inhibitor having an α,β-unsaturated ketone moiety, where the ketone has a sterically accessible electrophilic β-carbon, where the agent is cell permeable and active in intact cells, and where the agent is not a cyclopentenone prostaglandin of the J family, together with a pharmaceutically acceptable carrier, excipient, or diluent. The composition may further comprise an effective amount of at least one additional pharmaceutically active antineoplastic agent. The additional antineoplastic agent may be, for example, a topoisomerase 2 inhibitor, a DNA methyltransferase inhibitor, a topoisomerase 1 inhibitor, and a cyclopentenone prostaglandin of the J series. Other examples of the additional agent include etoposide, decitibine, and an active camptothecin analog.

In accordance with another aspect of the invention there are provided methods of treating additional diseases in a patient suffering from such a disease by administering to the patient an effective amount of a ubiquitin isopeptidase inhibitor. The diseases that may be treated in this manner include, for example, dry eye disorders, restenosis, inflammation, an autoimmune disease, ischemia, cachexia and/or muscle wasting.

In accordance with yet another aspect of the invention there is provided a method of stimulating growth of bone of hair in a patient, comprising administering to the patient an effective amount of a ubiquitin isopeptidase inhibitor.

In accordance with still another aspect of the invention there is provided a method of preventing of retarding graft rejection in a patient, comprising administering to the patient an effective amount of a ubiquitin isopeptidase inhibitor.

### BRIEF DESCRIPTION OF THE DRAWINGS

FIG. 1 shows the structures of (i) 2-cyclopenten-5-methylene-1-one; NSC-156236 (ii); PGB₁ (iii); 2,6-Diphenyl-4H-thiopyran-4-one [DPTP] (iv); NSC-302979 (v); Δ12-PGJ₂ (vi); dibenzylideneacetone [DBA] (vii.); and curcumin (viii).

FIG. 2 shows the effects of compounds on ubiquitin isopeptidase activity in colon cancer cells. A) Inhibition of isopeptidase proteolysis of Ub-PEST substrate. RKO and HCT 116 cells were treated with DMSO vehicle, 6, 20, or 60 μM of Δ12-PGJ₂, DBA, NSC-302979, or curcumin, 60 μM PGB₁, 60 μM NSC-156236, or 60 μM DPTP for 12 h, 37° C. Cell lysates from each treatment were incubated with Ub-PEST, as described in materials and methods. Samples were fractionated by SDS-PAGE and proteins detected with ubiquitin epitopes immunochemically (HCT 116 ^{+/+} western blot shown as example of raw data). Lane 1 shows the substrate prior to incubation with lysate. B) Inhibition of isopeptidase activity by test panel compounds. Isopeptidase activity was determined by measuring via densitometry, the amount of Ub generated by isopeptidase cleavage of Ub-PEST. Bar graph patterns are DMSO vehicle (black), Δ12-PGJ₂ (hatched), DBA (horizontal lines), NSC-302979 (white), PGB₁ (diagonally downward right lines), and NSC-156236 (diagonally downward left lines). The amount of Ub generated in the vehicle treated sample (panel A, lane 2) is arbitrarily designated 100%.

FIG. 3 shows the effects of compounds on ubiquitin isopeptidase activity in colon cancer cells. RKO (upper) and HCT 116 ^{+/+} cells (lower) were treated with DMSO vehicle, 6, 20, or 60 μM of Δ12-PGJ₂, DBA, NSC-302979, or curcumin, 60 μM PGB1, 60 μM NSC-156236, or 60 μM DPTP for 12 h at 37° C. Cell lysates (0.5 mg/mL) from each treatment were incubated with the isopeptidase substrate, z-LRGG-AMC, for 3 h, 37° C. The amount of AMC cleaved by isopeptidase was determined fluorometrically, as described in Materials and Methods.

FIG. 4 shows the effects of compounds on ubiquitin-dependant proteolysis in colon cancer cells. A) Accumulation of polyubiquitin. RKO-E6 cells were treated with DMSO vehicle for 24 h, 37° C., or with the concentrations of test compounds that gave 80% cell death (as determined by cell viability assay at 48 h) for 6, 12, or 24 h, 37° C. (EC₈₀: Δ12-PGJ₂ (13.2 μM), DBA (12.0 μM), NSC-302979 (3.5 μM), or curcumin 17 μM. Compounds that were inactive in the cell viability assay were treated with the highest concentrations examined (60 μM PGB₁, NSC-156236, and DPTP for 24 h, 37° C.). Cell lysates were fractionated by SDS-PAGE and proteins detected with ubiquitin epitopes immunochemically. B) Accumulation of p53, a protein targeted for proteasomal degradation. i. Schematic depicting p53 degradation via the proteasome pathway in RKO cells versus RKO-E6 cells. The activity of the HPV E6 oncoprotein hastens p53 degradation in RKO-E6 cells. ii. RKO and RKO-E6 cells were treated with vehicle, etoposide (50 μM), MG115 (20 μM), Δ12-PGJ₂ (60 μM), NSC-302979 (20 μM, DBA (20 μM), curcumin (60 μM), PGB1 (60 μM), NSC-156236, (60 XM)or DPTP 60 μM for 6 h, 37° C. Lysates were fractionated by SDS-PAGE and their p53 content determined immunochemically. iii. p53 levels were measured by densitometry and the ratio of p53 protein in RKO-E6 cells/RKO cells calculated.

FIG. 5 Reversibility of isopeptidase inhibition by Δ12-PGJ₂. Cell lysates (0.5 mg/mL) were treated with DMSO vehicle or 100 μM .12-PGJ₂ for 1 hour,25° C. The treated lysates were divided into two aliquots. One aliquot was immediately analyzed for isopeptidase activity by incubating with z-LRGG-AMC as previously described (Pre-Dialysis). The second aliquot was dialyzed by washing with 3 volumes of assay buffer on a centricon YM-30 column, followed by analysis for isopeptidase activity by incubating with z-LRGG-AMC (Post-Dialysis).

FIG. 6 shows cytotoxicity of compounds in HCT 116 and RKO cell lines with different p53 status. RKO and RKO-E6 cells (left column), and HCT116 p53 +/+ and HCT116 p53 −/− cells (right column) were incubated with vehicle or 0.5-60 μM NSC-302979, DBA, 0.12-PGJ2, NSC-156236, PGB_{B}1, curcumin, or DPTP for 48 h, 37° C. Cell viability was measured with MTT reagent as described in materials and methods. Data are percent of control viability, mean±S.D., ,n=4.

FIG. 7 shows cytotoxicity of the calibration set in HCT 116 and RKO cell lines with different p53 status. RKO and RKO-E6 cells (left column), and HCT116 p53 ^{+/+} and HCT116 p53 ^{-/-} cells (right column) were incubated with vehicle, 0.01- 100 μM etoposide, 0.001-1 μM paclitaxel, or 0.02-2 μM MG115 for 48 h, 37° C., and determined their effect on cell viability, as described in materials and methods.

### DETAILED DESCRIPTION

Abbreviations: DBA, dibenzylideneacetone; DMEM, Dulbecco's minimum essential medium; NCI DTP, National Cancer Institute Developmental Therapeutics Program; ECL, enhanced chemiluminescence; HRP, horse radish peroxidase; MG 115, carbobenzyloxy-L-leucyl-L-leucyl-norvaline; MTT, (3-(4,5-dimethylthiazo)-2-yl)-2,5-diphenyltetrazolium bromide; PBS, phosphate buffered saline; PG, prostaglandin; PVDF, polyvinyldifluoride; TBS, tris-buffered saline; UB, ubiquitin; z-LLVY-MCA, succinyl-L-leucyl-L-leucyl-L-valyl-L-tyrosine a-(4methyl-coumaryl-7-amide); z-LRGG-MCA, carbobenzoxy-L-leucyl-L-arginyl-Lglycyl-L-glycine a-(4-methyl-coumaryl-7-amide); DPTP, 2,6-Diphenyl-4H-thiopyran-4-one.

New Isopeptidase Inhibitors

Novel methods of inhibiting ubiquitin isopeptidases are provided that employ compounds that are chemically unrelated to prostaglandins of the J series. The inhibitors contain a pharmacophore that comprises an α,β-unsaturated ketone moiety having an electrophilic and sterically unhindered β carbon and that confers inhibitory activity toward isopeptidases. The activity of the inhibitors can be enhanced by the presence of an electron withdrawing group, for example, a halide atom or nitro group, at the α carbon of the unsaturated ketone. The ketone moiety may be a cross-conjugated α,β-unsaturated ketone that has one or two sterically accessible β-carbons. In addition, derivatives of such compounds are also active as isopeptidase inhibitors provided that the electrophilic β carbon is retained. Thus, for example, the double bond of the unsaturated ketone may be converted to an epoxide while retaining inhibitory activity. In the cross-conjugated molecules, one of both of the double bonds may be converted to epoxides while retaining the desired inhibitory activity. As example of an inhibitor containing an epoxide moiety is shikoccin epoxide and derivatives.

By disrupting ubiquitin salvage and impairing protein turnover, the inhibitors induce apoptosis independently of tumor suppressor p53 transactivation. The compounds are cell permeable and are orally active. Methods of making such compounds are known in the art. The present invention excludes use of cyclopentenone prostaglandins of the J series for inhibiting ubiquitin isopeptidases except that such prostaglandins may be used in combination with other inhibitors containing the pharmacophore.

In one embodiment, the ubiquitin isopeptidase inhibitors contain a conjugate cyclopentenone moiety. The α carbon of the cyclopentenone preferably contains an electron withdrawing group such as a halogen atom, a nitro group, a nitrilo group, or a carboxyl moiety such as a carboxylic acid, ester, or amide. Specific examples of such compounds include the punaglandins, such as PNG 2, PNG 3, PNG 4, Z-PNG 4 and PNG 6, whose structures are shown below.

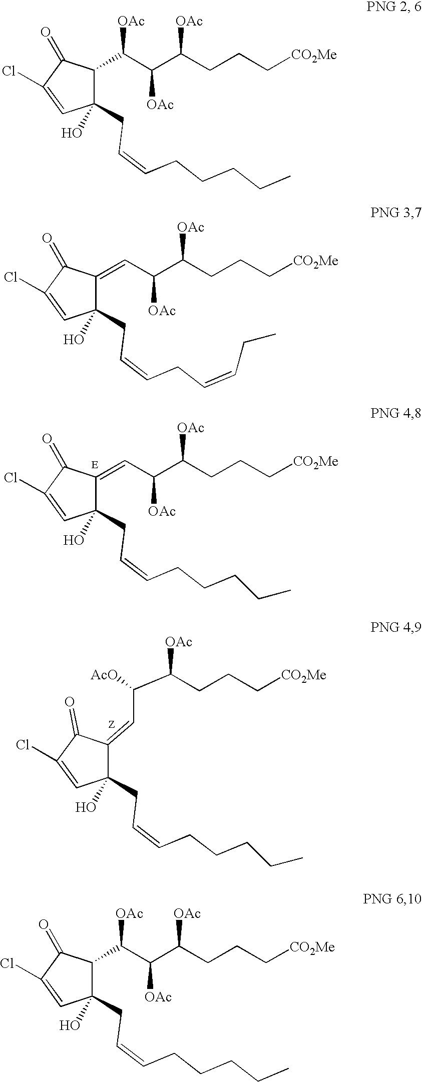

In another embodiment, the inhibitor is selected from the group consisting of cross-conjugated α,β-unsaturated ketones having two sterically unhindered β carbons. The ketone may be a cross-conjugated cyclopentadienone, such as the the diterpene shikoccin (NSC-302979), or may be an acyclic compound such as dibenzylideneacetone (DBA), or curcumin. The skilled artisan will recognize that additional compounds containing the pharmacophore also will be active as isopeptidase inhibitors, for example, rabdolatifolin and shikodomedin (Paquette et al., J. Amer. Chem. Soc. 118:11990 (1996); Id., 119:9662 (1997)) and O-methyl shikoccin. Shikoccin epoxide and O-methyl shikoccin epoxide (where the endocyclic double bond is replaced by an epoxide) also inhibit endopeptidase activity. In shikoccin epoxide and related compounds the β-carbon still is electrophilic by virtue of the strain of the epoxide ring.

The skilled artisan will further recognize that derivatives of these compounds that retain the pharmacophore identified above also will be active as isopeptidase inhibitors. These compounds may be used alone or in combinations, and may also be used in combination with cyclopentenone prostaglandins of the J series. These compounds are known in the art or can be prepared using known methods. See, for example, Paquette et al., supra, which describe methods for preparing shikoccin-type compounds and derivatives.

The present invention demonstrates that the ubiquitin dependent proteasome pathway contains molecular targets suitable for antineoplastic drug discovery (Kisselev and Goldberg, 2000). DBA, curcumin, and NSC-302979 inhibit the proteasome pathway in a manner chemically and mechanistically distinct from lactacystin (Fenteany et al., 1995), eponemycin (Meng et al., 1999), and peptide-aldehyde or boronate inhibitors (Adams et al., 1999), which all covalently inhibit the 20S catalytic subunit of the proteasome.

The present inventors have shown that members of the punaglandin family, shikoccin, dibenzylideneacetone, and curcumin inhibit cellular isopeptidases, and thereby cause cell death independently of p53 in isogenic pairs of RKO and HCT 116 cells with differential p53 status. The sesquiterpene, achillin, and 2,6-Diphenyl-4H-thiopyran-4-one, which have cross-conjugated dienones with sterically hindered electrophilic β-carbons, do not inhibit isopeptidases or cause significant cell death.

Furthermore, a catalytic-site proteasome inhibitor causes cell death independently of p53. The instant invention verifies a p53-independence of cell death caused by inhibitors of the proteasome pathway and demonstrates that the ubiquitin-dependent proteasome pathway contains molecular targets suitable for antineoplastic drug discovery.

The compounds described above may also be used in combination with other agents to achieve enhanced therapeutic results. For example, the compounds can be used in combination with agents that depend on the integrity of the proteasome activity for repair of DNA damage, for example a topoisomerase 2 inhibitor such as etoposide, a DNA methyltransferase inhibitor such as decitibine, and a topoisomerase 1 inhibitor such as an active camptothecin analog.

Targeting of Inhibitors

In an embodiment one or more inhibitors are targeted to a tissue such as a tumor for increased efficacy. The tissue may be an overgrowth of a normal tissue, such as a hyperactive thyroid or an oversized prostate gland. A particular advantage of this embodiment of the invention is that the pharmaceutical can be delivered directly to the unwanted tissue at a known amount, allowing a controlled destruction of the tissue. Moreover, in a desirable embodiment a natural apoptosis event occurs, which facilitates absorption of destroyed tissue by surrounding healthy tissue. In an embodiment where only limited destruction is desired, preferably after physically delivering the pharmaceutical to a target tissue and allowing the active agent to bind up the ubiquitin isopeptidase, a second quench reagent, such as a reduced glutathione or other physiologically acceptable nucleophile is added to the region, and reacts with any remaining compound there. Physical delivery may be achieved by, for example injection, catheter introduction as, for example, reviewed in U.S. Pat. No. 6,369,030.

In a particularly desirable embodiment the active agent is delivered to a specific undesirable tissue type such as a tumor (for cancer therapy) or gamete producing cells (for prevention of pregnancy). A variety of methods are known for such delivery. For example, an antibody (whole antibody, chimeric antibody, antibody fragment, or other binding molecule) that specifically binds to gamete producing cells, egg cells, prostate tissue cells, or tumor cells may be labeled with active agent and then introduced into the body, to allow the binding molecule with attached active agent to diffuse to and concentrate at the targeted site. Other binding systems are well known and contemplated such as the use of biotin-avidin as exemplified in U.S. Pat. No. 5,630,996.

In a related embodiment the active agent is prepared as a solution, precipitate, or suspension within a particle such as a lipid vesicle and the particle is delivered to the site, allowing a targeting binding molecule on the particle surface to bind to specific ligand(s) expressed on cell surfaces there. The binding molecule preferably is covalently conjugated to the active agent or to a particle that contains the active agent. A variety of cell specific binding substances such as humanized antibodies specific for tumor cell surface expressed antigen are known and can be used. See for example U.S. Pat. No. 6,521,211, which describes the use of targeted vesicle compositions. Also see U.S. Pat. No. 6,284,280, which describes the use of microparticles that controllably can release their contents using ultrasound.

In another embodiment suitable for targeting tumors, the active agent is physically mixed with or covalently bound to porphyrins (such as photophrin) that are used to localize to tumors for photodynamic therapy. In a related embodiment, the covalent linkage of the active agent to porphyrin itself is light sensitive. The latter embodiment may be used by a method wherein the active agent-porphyrin conjugate is added, for example by injection near to a tumor site. The porphyrin is taken up and concentrates within tumor cells. Then strong light irradiates the tumor site and kills cells by regular photodynamic therapy. An advantage of this procedure is that the added active agent can greatly facilitate normal tumor cell killing in response to the light. According to an embodiment, the active agent is added to a material used in photodynamic therapy, either by covalent conjugation or by physical admixture and improves the efficacy of that therapy.

Other delivery systems useful for the pharmaceutical preparations described herein include for example biodegradable polymeric matrixes to which active substances may be loosely attached or covalently attached, for gradual release at or near a target tissue. See for example U.S. Pat. Nos. 6,030,941 and 5,626,862, which specifically describe chemicals and methods of their use, and which more specifically is incorporated by reference. In a related embodiment, a chemical conjugate including a first binding moiety, such as epidermal growth factor, which preferentially binds to a tumor cell, and is internalized by the cell, may be used with a second moiety linked to the first moiety, and comprising a biodegradable polymeric carrier, such as polyglutamic acid, to which one or more active agent molecules as described herein, are attached. The degradation of the carrier by intracellular enzymes releases the active agent, resulting in selective destruction of the tumor cells. See for example U.S. Pat. Nos. 5,087,616 and 4,356,166 the contents of which are incorporated by reference in their entireties. In yet another embodiment, a nutritive factor is used to carry the active agent into a cell. For example, the active agent may be coupled to ceruloplasmin, which is used for delivery of copper into cells, or to the selenium transport protein, which is used for delivery of selenium into cells. In a desirable embodiment the active agent is linked to a metabolite that is limited to specific tissue intake, for improved targeting.

Pharmaceutical Compositions of the Inhibitors

Pharmaceutical compositions of this invention comprise at least one compound having ubiquitin isopeptidase activity that comprises an α,β-unsaturated ketone moiety having an electrophilic and sterically unhindered β carbon. As described above, the activity of the inhibitors can be enhanced by the presence of an electron withdrawing group, for example, a halide atom or nitro group, at the α carbon of the unsaturated ketone. The ketone moiety may be a cross-conjugated α,β-unsaturated ketone that has one or two sterically accessible β-carbons. The compound may also be a suitable derivative of such compounds that is active as isopeptidase inhibitors provided that the electrophilic β carbon is retained. Thus, for example, the double bond of the unsaturated ketone may be converted to an epoxide while retaining inhibitory activity. In the cross-conjugated molecules, one of both of the double bonds may be converted to epoxides while retaining the desired inhibitory activity. An example of an inhibitor containing an epoxide moiety is shikoccin epoxide and derivatives. The inhibitor compound may be present as a pharmaceutically acceptable salt where appropriate, for example when the inhibitor contains a carboxylic acid or amino function. Suitable pharmaceutically acceptable salts are well known in the art.

The inhibitor is present in the composition together with a pharmaceutically acceptable carrier, adjuvant or vehicle. The compositions may include more than one inhibitor compound and/or its corresponding salt, in any proportion. The inhibitor compounds may also be combined with cyclopentenone prostaglandins of the J series, though the invention excludes use of J series prostaglandins in the absence of at least one other isopeptidase inhibitor. The compositions may also include other pharmaceutically active components, such as other antineoplastic agents that are directed at targets other than ubiquitin isopeptidase. Suitable antineoplastic agents to be used in such combinations include a topoisomerase 2 inhibitor such as etoposide, a DNA methyltransferase inhibitor such as decitibine, and a topoisomerase 1 inhibitor such as an active camptothecin analog.

The skilled artisan will recognize that a wide variety of additional antineoplastic agents also are known and are suitable for use in combination with active agents described herein for the present invention. An effective amount is used for delivery to one or more tissues that have ubiquitin isopeptidase activity sensitive to the active agent. For overactive tissues that need to be partially removed, an effective amount will be appreciated by the clinical determination in each instance. Generally, an effective amount contains a quantity of an active component that is sufficient to produce a therapeutically desirable result Methods of determining an effective amount are known in the art.

Generally, an effective amount of active agent to be added to an individual will vary greatly depending on the tissue to be treated and the mode of treatment However, it is desired to add enough active agent to the tissue to achieve serum concentrations or intracellular concentrations of at least 0.1 micromolar, 0.3 micromolar, 1 micromolar, 5 micromolar, 20 micromolar, 75 micromolar or higher. Preferably, depending on the type of agent, between 10 and 1,000 micromolar (tissue final concentration) may be delivered. When added as a systemic or as a chronic infusion, a suitable dosage for use in the treatment of an ubiquitin isopeptidase responsive medical condition, the active agent may vary between 0.0005 mg/kg to about 10 mg/kg body weight, in particular between 0.005 mg/kg to 1 mg/kg body weight, depending upon the specific condition to be treated, the age and weight of the specific patient, and the specific patient's response to the medication. The exact individual dosage, as well as the daily dosage, will be determined according to standard medical principles under the direction of a physician In an embodiment, enough is added to inhibit 50% of the ubiquitin isopeptidase activity (measured by taking a biopsy, immediately freezing, and determining enzyme activity from thawed samples) within 1 hour of administration.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. Preferably, administration is by oral administration or administration by injection. The pharmaceutical compositions of this invention may contain any conventional non-toxic. pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents, such as, for example, Tween 80, and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions and emulsions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-tansdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Dosage levels of between about 0.01 and about 100 mg/kg body weight per day, preferably between about 0.5 and about 50 mg/kg body weight per day of the active ingredient isopeptidase inhibitor compound are useful in the prevention and treatment. Typically, the pharmaceutical compositions of this invention will be administered from about 1 to about 5 times per day or alternatively, as a continuous infusion. The amount of active ingredient that may be combined with the carrier materials to produce a single dosage form will vary depending upon the patient treated and the particular mode of administration. A typical preparation will contain from about 5% to about 95% active compound (w/w). Preferably, such preparations contain from about 5% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease or oncological malady, the patient's disposition to the disease or oncological malady and the judgment of the treating physician.

A patient is a mammal, preferably a human

Treatment of Other Diseases

As illustrated above, the isopeptidase inhibitors described herein act as inhibitors of the proteasome pathway/ Accordingly, the skilled artisan will recognize that these inhibitors also may be used to treat other disease processes that are mediated by the proteasome pathway. The ubiquitin-proteasome pathway plays a pivotal role in the degradation of short-lived and regulatory proteins important in a variety of basic cellular processes, including regulation of the cell cycle, modulation of cell surface receptors and ion channels, and antigen presentation. The pathway involves an enzymatic cascade through which multiple ubiquitin molecules are covalently attached to the protein substrate, which is then degraded by the 26S proteasome complex. Various components of this pathway have been implicated in several forms of malignancy, in the pathogenesis of several genetic diseases (including cystic fibrosis, Angelman's syndrome, and Liddle syndrome), in immune surveillance/viral pathogenesis, in the pathology of muscle wasting as reviewed, for example, in “The ubiquitin-proteasome pathway and pathogenesis of human diseases” by Schwartz and Ciechanover Annu. Rev. Med. 50: 57-74 (1999); in the pathology of psoriasis, restenosis, proliferative diseases generally, inflammatory, autoimmune and ischemia responses, multiple myeloma, HIV replication, bone growth, hair growth and muscle wasting.

Accordingly, the skilled artisan will appreciate appropriate vehicles, modes of delivery, effective amounts and related methods for using one or more active agents defined herein to treat hair loss, memory loss, bone loss, AIDS, autoimmune disease, cancers (in particular multiple myeloma), and diseases of the eye. Each of these diseases may be prevented, cured and/or alleviated by the use of one or more pharmaceutical preparations as described herein.

A skilled artisan informed by a reading of the specification will comprehend use of the active agents described herein in a number of ways to prevent or ameliorate diseases mediated by proteasomes. For example, the use of proteasome inhibitors to treat dry eye disorders and other disorders that involve wetting of the eye has been describe. See WO02094311A1, which is hereby incorporated by reference. Accordingly the isopeptidase inhibitors describe herein may be used to treat dye eye disorders and the like, alone or in combination with the compounds described in WO02094311A1.

Similarly, proteasome inhibitors have been used to prevent the complications of cell proliferation associated with restenosis of blood vessels after angioplasty, especially of smooth muscle cells within blood vessel walls. See WO02060341A2, which is hereby incorporated by reference. Accordingly the isopeptidase inhibitors described herein may be used to treat or prevent restenosis, alone or in combination with the compounds described in WO02060341A2. In a desirable embodiment, an active agent is bound to the surface of a stent and the stent planted in a blood vessel after angioplasty. Many of the active agents are hydrophobic enough, or can be made hydrophobic enough by derivitization with aromatic, aliphatic or other residue(s) so that the agent binds non-covalently to a stent having a surface that binds hydrophobic materials well, such as a plastic polymer. Preferably, the active agent is adsorbed onto the stent surface(s), optionally rinsed, and then the stent is inserted. Active agent slowly desorbs from the stent surface over time. In another embodiment, the active agent is covalently bound to the surface via a bond or intermediary moiety that slowly hydrolyzes in the presence of water, releasing the active agent over a long time period.

These two slow release options may be used for other materials that are implanted, for example into tumors or other areas of undesirable hyperplastic growth. In an embodiment, this slow release method is used to improve antitumor therapy based on implantation of devices such as Gliadel™ wafers, which release the anti-cancer agent carmustine slowly into a brain tumor site. By adding an active agent as described herein that inhibits proteasomes, such present anti-cancer treatments can be improved. Use of this second agent can yield improvements in clinical outcome of at least 10%, 20% 30% or even greater, as measured by mean percent survival time increases. That is, the added use of an active agent can increase the fraction of tumor cells that can be killed by other treatments such as DNA damaging therapeutic agents (such as carmustine), and specifically is intended to complement and improve outcomes for radiation treatment and anti-tumor drug treatment. The active agent can be combined with other agents that cause to degradation of Cdc25A as described in WO0166708A2.

An active agent as described herein also can be used to replace or augment, and thereby provide synergy with other inhibitors of proteasomal activity. Proteasome inhibitors that already are known can be replaced or augmented in known methods using the ubiquitin isopeptidase inhibitors described herein. For example, inhibitors and uses for ameliorating diseases are described in WO0128579A2, including compounds that inhibit the activity of NF-κB or inhibit the activity of the proteasome or both and thereby promote bone formation and hair growth. These materials and methods further are useful in treating osteoporosis, bone fracture or deficiency, primary or secondary hyperparathyroidism, periodontal disease or defect, metastatic bone disease, osteolytic bone disease, post-plastic surgery, post-prosthetic joint surgery, and post-dental implantation. Additionally such active agent compounds described herein also may be used to stimulate the production of hair follicles and are thus useful in stimulating hair growth, including hair density. Other inhibitors and methods of their use, are contemplated as well, as for example describe in US20020049157A1, including lactacystin, DPBA and their analogs. The active agents described herein may replace or augment such inhibitors in compositions for the following additional purposes: (1) to disrupt mitochondrial function (useful against cancer, inflammation, adverse immune reaction and hyperthyroidism), (2) to disrupt nitric oxide synthesis (useful against inflammation and septic shock), and (3) to reverse ongoing adverse immune reactions, such as autoimmune diseases and graft rejection. In the latter case, the compositions preferably are administered after activating a patients' T cells. In this context, active agent also can be combined with immuno-suppressinve drugs such as rapamycin, cyclosporin A and FK506.

An active agent as described herein may replace or augment an anticancer agent containing a topoisomerase activity inhibitor and thereby inhibit the proteasome promotion of topoisomerase degradation This inhibition provides a sufficient amount of topoisomerase in cancer cells, thereby potentiating anticancer agents that contain topoisomerase activity inhibitors. Similarly, combined use of a proteasome activity inhibitor such as an active agent as described herein, with DNA strand-injury type anticancer agents (for example, platinum complexes, alkylating agents, bleomycins), which directly act on DNA but suffer from regulation or reduction in the effects thereof by the proteasome activity, can potentiate the effects, as described, for example in WO0047230A1.

Active agents described herein may be used to specifically modulate cellular pathways, immunity and therapies associated with these, as for example, describe in WO0033654A1. In one embodiment the active agent is used to complement or replace an inhibitor such as another proteasome inhibitor or an HIV protease inhibitor described in this reference. Such an inhibitor may be, for example, a serine protease or cysteine protease inhibitor that modulates cellular pathways such as those involved in cell activation, metabolism, proliferation, differentiation, maturation, cycle, and death. This use is especially relevant for cancer treatment, allergy, vaccines, autoimmune disorder, inflammation, transplant, burn, trauma, acute ischemia, stroke, aging, wasting syndrome, and infectious conditions. Further compounds that may be replaced or augmented inhibit the activity of NF-κB or inhibit the activity of the proteasome or both and promote bone formation and hair growth and are thus useful in treating osteoporosis, bone fracture or deficiency, primary or secondary hyperparathyroidism, periodontal disease or defect, metastatic bone disease, osteolytic bone disease, post-plastic surgery, post-prosthetic joint surgery, and post-dental implantation as for example, describe in US20020107203A1. Such use also can stimulate the production of hair follicles and thereby stimulate hair growth, including hair density.

Further uses include the treatment of inflammatory and autoimmune diseases by administering an active agent describe herein, optionally in combination with other ubiquitin pathway inhibitor(s), agent(s) that interfere with the activation of NF-κB via the ubiquitin proteasome pathway, or mixtures thereof. The treatment of inflammatory and autoimmune disease may be carried out by administering an effective combination of a glucocorticoid and an active agent, ubiquitin pathway inhibitor, agent that interferes with the activation of NF-κB via the ubiquitin proteasome pathway, or mixture thereof. Pharmaceutical compositions comprising a combination of a glucocorticoid and an active agent, ubiquitin pathway inhibitor, agent that interferes with the activation of NF-κB via the ubiquitin proteasome pathway, or mixture thereof are also contemplated. Such agonists may be for example found in US20010051654A1. Still further agonists for co-administration, methods and diseases that can be addressed by active agents and methods of their use as describe herein can be found, for example in U.S. Pat. No. 5,985,824; US20030069189A1; U.S. Pat. No. 6,271,199; U.S. Pat. No. 6,287,858; U.S. Pat. No. 6,051,684; U.S. Pat. No. 6,548,668; U.S. Pat. No. 5,693,617; U.S. Pat. No. 5,847,076 and U.S. Pat. No. 5,340736. The contents of each patent and patent application cited herein, and more specifically the chemical agents describe in every cited reference therein having an effect on a biochemical system, the method of using the chemical agents for their effects, and the treatment of disease with those agents as described in each reference most specifically are incorporated by reference.

The compounds of this invention are also useful as commercial reagents which effectively bind to aspartyl proteases, particularly ubiquitin isopeptidases. As commercial reagents, the compounds of this invention, and their derivatives, may be used to block proteolysis of a target peptide or may be derivatized to bind to a stable resin as a tethered substrate for affinity chromatography applications. These and other uses which characterize commercial aspartyl protease inhibitors will be evident to those of ordinary skill in the art.

### EXAMPLES

Experimental Procedures

Materials:

Δ(Delta)12-PGJ2 and MG131 (Cayman Chemicals, Ann Arbor, Mich.); dibenzylideneacetone, etoposide, paclitaxel, Curcumin, and 2,6-Diphenyl-4H-thiopyran-4-one (Sigma, St. Louis, Mo.); NSC-302979 and NSC-156236 (Drug Synthesis and Chemistry Branch, Developmental Therapeutics Program, Division of Cancer Treatment, National Cancer Institute); complete protease inhibitor mixture (Roche Molecular Biochemicals, Indianapolis, Ind.); enhanced chemiluminescence reagents (Amersham Pharmacia, Piscataway, N.J.); antibodies directed against p53 (DO-1), horseradish peroxidase-conjugated secondary antibodies (Santa Cruz Biotechnology, Santa Cruz, Calif.), and ubiquitin (Ubi-1)(Zymed Laboratories, Inc., San Francisco, Calif.); MG 115 (Peptides International, Louisville, Ky.); z-LLVY-MCA, z-LRGG-MCA (Biomol Research laboratories, Plymouth Meeting, Pa.); MTT (3-(4,5-dimethylthiazo)-2yl)-2,5-diphenyltetrazolium bromide (Molecular Probes Inc, Eugene, Oreg.); Ub-PEST (gift of Dr. Martin Rechsteiner, Department of Biochemistry, University of Utah); Centricon YM-30 centrifugal filters (Amicon Bioseparations, Millipore, Bedford, Mass.).

Cell Culture: RKO and RKO-E6 colon cancer cells were a gift from Dr. Mark Meuth, Institute for Cancer Studies, University of Sheffield, Sheffield, U.K. HCT 116 colon cancer cells with varying degrees of p53 haplosufficiency were a gift of Dr. Bert Vogelstein. RKO and RKO-E6 cells were maintained in DMEM (supplemented with 2 mM L-glutamine, 1 mM sodium pyruvate, 50 units/ml penicillin and streptomycin, and 10% (v/v) FBS) in a humidified incubator with 5% CO₂. HCT 116 were maintained cells in McCoy's 5A medium (supplemented with 1 mM sodium pyruvate, 50 units/ml penicillin and streptomycin, and 10% (v/v) FBS) in a humidified incubator with 5% CO₂.

Immunochemical Detection of Proteins

The medium was removed and cells lysed in 50 mM Tris, pH 7.4, 100 mM NaCl, 2 mM EDTA with 0.1% SDS, 0.1% deoxycholate, 1× complete protease inhibitor mixture. Protein concentration was measured by the Bradford method. Equal portions of the total cell lysate were fractionated from each sample (12.5 μg of protein) by SDS-PAGE. Proteins were transferred to poly(vinylidene difluoride) blocked with 5% (w/v) nonfat dry milk in Trisbuffered saline [20 mM Tris-HCl, pH 7.5, 100 mM sodium chloride, 0.1% (v/v) Tween 20]. Proteins were detected immunochemically by using primary antibodies directed against p53 (1:4000) or ubiquitin (1:1000), followed by horseradish peroxidase-conjugated secondary antibodies (1:4000). Antigen-antibody complexes were detected with enhanced chemiluminescence reagents. Gels were scanned and quantified intensities using Kodak 1D Image Analysis Software.

Cell Viability Assay

Cell viability was detected by the MTT assay. Briefly, 1×10⁵ cells were incubated per well of a sterile, 96-well assay plate with 0-60 μM of test compounds for 48 hours. MTT reagent was added to each well, final concentration of 0.5 mg/mL, and incubated for an additional 3 hours. The media was aspirated and remaining MTT reagent from each well and added 100 μL of HCl:isopropanol (1:24). The absorbance of each sample was measured at 405 nm.

Ubiquitin Isopeptidase Activity Assays

Cellular isopeptidase enzymatic activity was measured with two assays that used different substrates. In one assay ubiquitin-PEST (Ub-PEST) was used, a full-length ubiquitin molecule with an 18 amino acid c-terminal peptide extension (total mass=10.5 kDa). Ubiquitin isopeptidases specifically cleave the 18 amino acid peptide extension, releasing full-length ubiquitin (8.5 kDa).

Briefly, 6×10⁵ cells were incubated with 0-60 μM of test compounds for 12 hrs. Cells were lysed in 50 μL of 25 mM HEPES, 5 mM EDTA, 0.1% CHAPS, 5 mM ATP, pH 7.5. The protein concentration of each sample was adjusted to 0.3 mg/mL and incubated with 50 μg/mL Ub-PEST for 45 minutes at 25° C. Under these conditions, Ub-PEST hydrolysis occurs at a linear rate. 20 μL samples were mixed with 20 μL 2× Laemmli buffer, boiled briefly, and fractionated by SDSPAGE. Isopeptidase activity was monitored by determining the amount of product (8.5 kDa ubiquitin) formation.

The second assay used a fluorescent tetrapeptide, z-LRGG-AMC, as a substrate that mimics the carboxy-terminus of ubiquitin. Isopeptidase activity hydrolyzes the bond between the c-terminal glycine and the fluorophore. This tetrapeptide also undergoes slow proteolysis by the catalytic subunit of the proteasome. To minimize this background rate of proteolysis cell lysates were incubated with 30 μM MG115 for 30 minutes at 4° C., prior to substrate incubation (>90% proteasome inhibition). Cells were teeated as above, lysed in 250 μL of lysis buffer per sample, and adjusted protein concentration to 0.5 mg/mL prior to incubation with MG115. Next, z-LRGG-AMC substrate was added and fluorescence was quantified of the AMC moiety cleaved by isopeptidase action.

Δ-12-PGJ₂ irreversibility was determined. Cell lysates were incubated with vehicle or 100 μM test compound and isopeptidase activity was measured fluorimetrically. 500 μl portion of each sample were dialyzed through a Centricon filter with a molecular weight cut-off of 25 kDa. After washing with 3 volumes of assay buffer, isopeptidase activity was measured in the filtrate. If filtration does not reverse inhibition, it implies that Δ12-PGJ₂ is an irreversible inhibitor.

Statistics

Analysis of variance (ANOVA) was used for statistical calculations.

Results

Sub-Structure Analysis of the NCI Cancer Screening Database.

The Developmental Therapeutics Program (DTP) of the National Cancer Institute (NCI) has systematically evaluated >70,000 compounds for cytostatic and cytotoxic activity against human cell lines in vitro (Monks et al., 1997). The cell lines typify cancers of the colon, blood (leukemia), brain, breast, kidney, lung, ovary, prostate and skin (melanoma). Intramural NCI investigators, who have access to the entire database, have applied this information-intensive approach with promising results (Weinstein et al, 1997; Shi et al 1998). Extramural investigators have access to a restricted portion of the database, last released in August 2000. Using these available data, a sub-structure search was conducted to identify suitable molecules to test whether a cross-conjugated α,β-unsaturated dienone with two sterically accessible β-carbons is a primary molecular determinant that permits inhibition of isopeptidases. Specifically, non-prostanoid compounds were sought with this feature that varied in the accessibility of their olefinic β-carbons (e.g. β-carbons with —H versus with —CH₃ substituents).

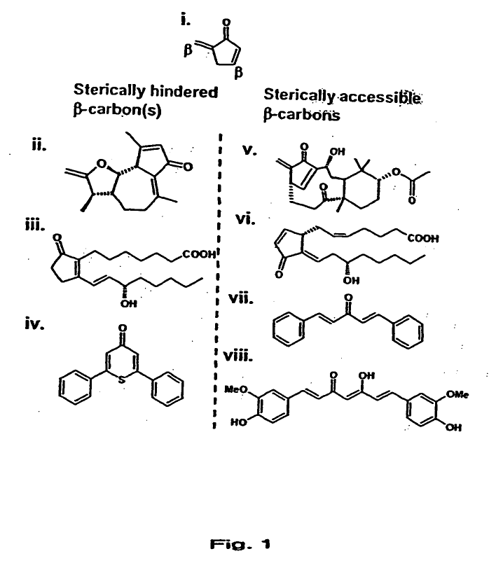

The substructure query, 2-cyclopenten-5-methylene-1-one [FIG. 1, i.], yielded eight compounds. All have a cyclic (bis) α,β-unsaturated ketone with one endo- and one exo-olefin. They are otherwise chemically unrelated to Δ12PGJ₂ or other PG. The NCI provided two of the eight compounds we requested for our experimental use: the sesquiterpene, NSC-156236 [FIG. 1, ii.] and the diterpene, NSC-302979 [FIG. 1, v.]. Like Δ12PGJ₂ [FIG. 1, vi.], the endo- and exo-olefins of NSC-302979 have sterically accessible carbons that can react with nucleophiles (e.g. cysteine; Rodriguez et al., 1997). Analogous to PGB₁ [FIG. 1, iii.], NSC-156236 has methyl-substituted β-carbons at the endo- and the exo-olefin of the dienone. These β-carbons are sterically hindered and therefore should not react readily with relevant physiological nucleophiles (Rodriguez et al., 1997). To further reinforce the pharmacophore hypothesis, several commercially available compounds were evaluated. Two of these compounds, dibenzylideneacetone [DBA, FIG. 1, vii.] and curcumin [FIG. 1, viii.], have sterically accessible β-carbons. The final compound, 2,6Diphenyl-4H-thiopyran-4-one [DPTP, FIG. 1, iv.], resembles DBA, except that it has a bulky sulfur atom sterically hindering its β-carbons. In summary, the pharmacophore hypothesis predicts that compounds ii. through iv. will be inactive as isopeptidase inhibitors and compounds v. through viii. will be active as isopeptidase inhibitors.

Electrophilic Cross-Conjugated Dienones Inhibit Cellular Ubiquitin Isopeptidases.

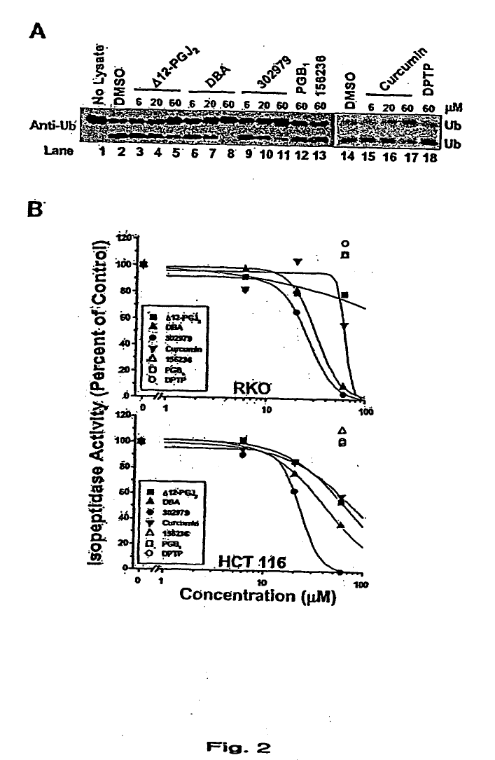

Little is known about the substrate specificity of the individual isopeptidase family members. To investigate total cellular isopeptidase activity, two simple substrates were used, Ubiquitin-PEST (Ub-PEST) and z-LRGG-AMC, which most isopeptidases use as substrates. FIG. 2A shows the effect of the test panel compounds, Δ12PGJ₂ [lanes 3-5], DBA [lanes 6-8], NSC-302979 [lanes 9-11], PGB₁ [lane 12], NSC-156236 [lane 13), curcumin [lanes 15-17], and DPTP [lane 18] on cleavage of the Ub-PEST substrate by isopeptidases in HCT 116 colon cancer cell lines (similar results were obtained for RKO cells; raw data not shown). Consistent with the pharmacophore hypothesis, the compounds with cross-conjugated ketones and sterically accessible β-carbons, Δ12PGJ₂, DBA, NSC-302979, and curcumin, each inhibited isopeptidase activity in a concentration dependent manner [FIG. 2B]. Compounds with sterically hindered β-carbons, PGB₁, NSC-156236, and DPTP did not inhibit isopeptidase activity. The rank order of potency for inhibition of ubiquitin-PEST hydrolysis by isopeptidases was DBA≈NSC-302979>Δ12PGJ₂>curcumin>>NSC-1 56236≈PGB₁≈DPTP.

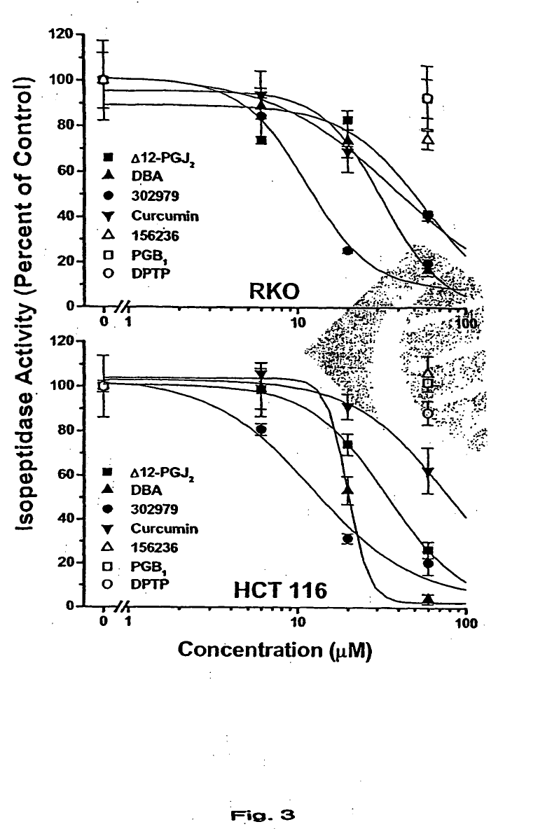

To verify the results with Ub-PEST in FIG. 2, z-LRGG-AMC was used as the substrate for isopeptidases [FIG. 3]. NSC-302979, Δ12PGJ₂, DBA, and curcumin each inhibited ubiquitin isopeptidase activity, while NSC-156236, PGB₁, and DPTP did not The rank-order of potency for inhibition of z-LRGG-AMC hydrolysis by ubiquitin isopeptidases was NSC-302979 >DBA>Δ12PGJ₂ >curcumin>>NSC-156236≈PGB₁≈DPTP.

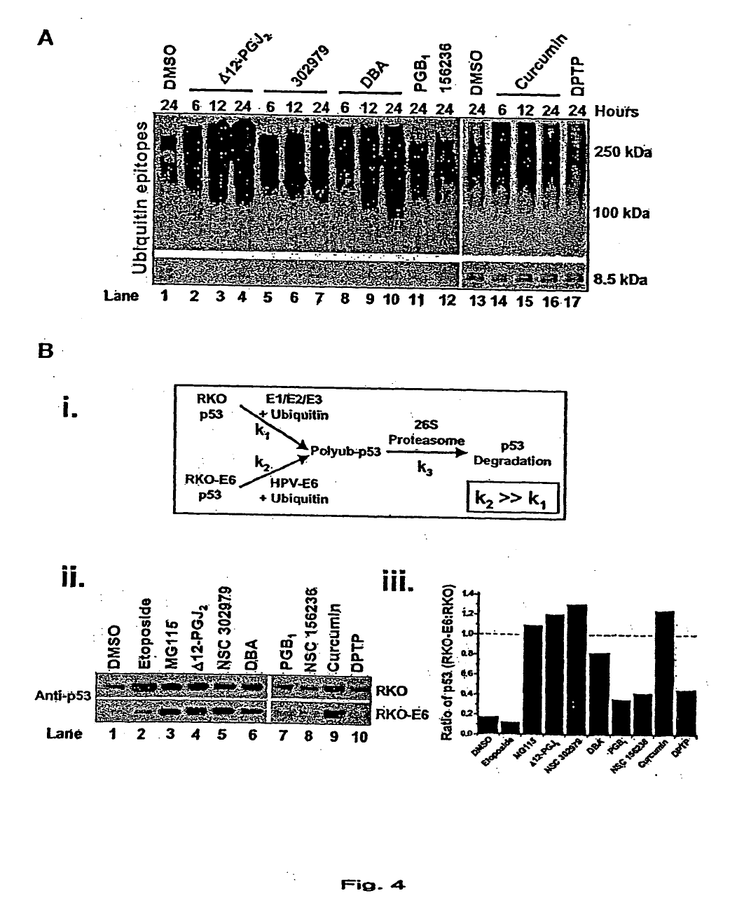

Consistent with inhibition of the isopeptidases that disassemble ubiquitin polymers and ubiquitin-protein conjugates, protein species with polyubiquitin conjugation accumulated in cells (e.g. in RKO-E6 cells, FIG. 4A) treated with Δ12PGJ₂ [lanes 2-4], NSC-302979 [lanes 5-7], DBA [lanes 8-10] or curcumin [panes 14-16]. PGB₁ [lane 11], NSC-156236 [Dane 12], and DPTP [lane 17], compounds that did not inhibit isopeptidase activity, did not cause appreciable cellular accumulation of ubiquitin conjugates.

As a consequence of polyubiquitin accumulation, monoubiquitin is initially depleted in cells treated with isopeptidase inhibitors [8.5 kDa band, FIG. 4A, lanes 2, 5, 8, and 14 vs. lanes 1 or 13]. It is predicted that this would affect the rate at which substrates are degraded via the ubiquitin-proteasome pathway. RKO and RKO-E6 cells allow a convenient test of the pharmacophore hypothesis with p53, a single, explicit substrate targeted for ubiquitin-dependent proteolysis. RKO-E6 cells, an isogenic variant of RKO cells, harbor the HPV-E6 oncoprotein that, together with E6-AP ubiquitin ligase, hastens proteasome-mediated degradation of p53 [FIG. 4B, i., Ashcroft and Vousden, 1999). Thus, under most conditions, the levels of p53 are significantly lower in RKO-E6 cells than in RKO cells (e.g. treatment of cells with vehicle or etoposide, a DNA damaging agent; FIG. 4B, ii., lanes 1 and 2, respectively, and FIG. 4B, iii.). One exception is when the ubiquitin-proteasome pathway is inhibited (e.g. treatment of cells with the proteasome inhibitor, MG 115; lane 3); then, accumulation of p53 in RKO-E6 and RKO cells equalizes. Accordingly, the ratio of p53 protein [RKO-E6 cells/RKO cells] approached unity in cells treated with the isopeptidase inhibitors, Δ12PGJ₂ [lane 4], NSC-302979 [lane 5], DBA [lane 6], and curcumin [lane 9]. Test compounds that did not inhibit ubiquitin isopeptidase activity [PGB1 (lane 7), NSC 156236 (lane 8), and DPTP (lane 10)] had p53 protein ratios (RKO-E6:RKO) significantly less than unity, suggesting that they do not inhibit p53 degradation via the ubiquitin-proteasome pathway. p53 accumulation caused by the pharmacophore test compounds is not a result of 20 S proteasome inhibition, as none of the compounds with cross-conjugated dienones inhibited the 20 S catalytic subunit of the proteasome under these conditions (Mullally et al., 2001 and data not shown). Despite its accumulation in the presence of Δ12PGJ₂, p53 is inactivated as a transcription factor under these conditions (Mullally et al., 2001); furthermore, p53 activation is insufficient to cause its stabilization in RKO-E6 cells, as the p53 protein ratio (RKO-E6:RKO=0.1) in etoposide treated cells fails to approach unity. Collectively, the data in FIGS. 2 through 4 affirm that the pharmacophore hypothesis extends beyond the prostanoid family into the diterpene and other chemical families.

The Prototype Isopeptidase Inhibitor, Δ12-PGJ₂, Inhibits Ubiquitin Isopeptidases Irreversibly.

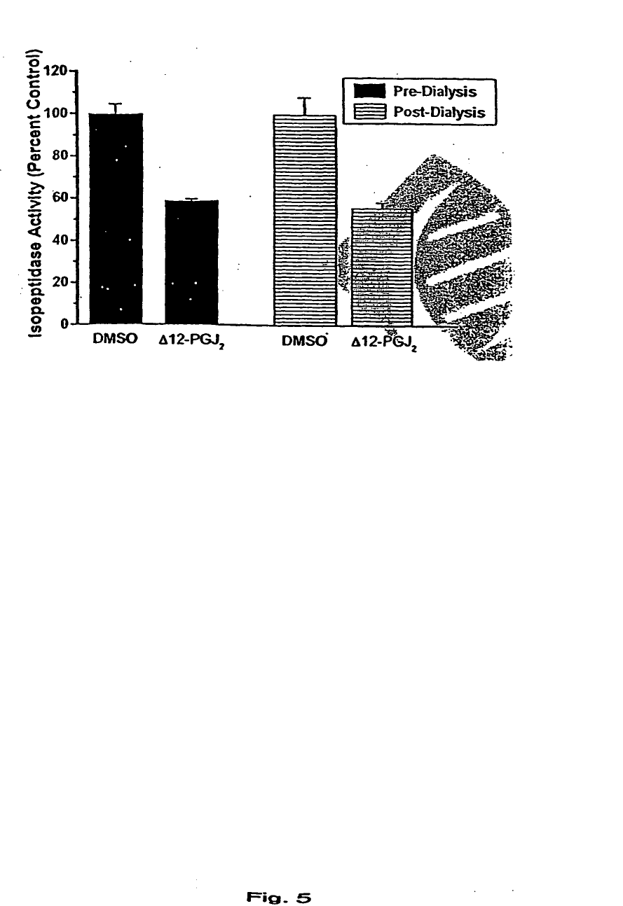

Although a covalent complex between an isopeptidase and one of the isopeptidase inhibitors has not been identified, this proposed mechanism of action is consistent with data obtained from analyzing cell lysates treated with Δ12-PGJ₂. Treatment of cell lysates (vs. treatment of whole cells) should exclude the likelihood of transcriptional translational events due to the nature of the lysate preparation (i.e. sonication of cell lysates likely shears all polynucleotides). Δ12-PGJ₂ inhibited isopeptidase activity in treated cell lysates [FIG. 5, pre-dialysis]. Furthermore, isopeptidase inhibition by Δ12-PGJ₂ could not be reversed by dialysis of treated lysates [FIG. 5, post-dialysis].

Inhibitors of Ubiquitin Isopeptidases Cause Cell Death Independently of Tumor Suppressor p53 Function.

Previously, the present inventors showed that electrophilic prostaglandins, typified by Δ12-PGJ₂, inhibit p53-mediated transcription under the same conditions in which they cause cell death, suggesting that cell death occurs independently of p53 (Mullally et al., 2001). Therefore, integration of the pharmacophore and molecular mechanism hypotheses predicts that various isopeptidase inhibitors will cause cell death independently of tumor suppressor p53 function Analysis of the NCI 60 cell line cancer screening data, according to O'Connor et al., 1997, showed that NCI-302979 and curcumin (NSC-32982) act independently of p53 [Table 1 below]. NSC-156236 had no appreciable cytotoxic activity. Thus, data on NSC-302979, NSC-32892 and NSC-156236, available from the DTP public database, fulfill the minimal, initial prediction of our hypotheses.

TABLE 1
Mean LC50 (uM) of NCI DTP Database Compounds Examined

| | Wild Type p53 | Mutant p53 | Statistical |
|---|---|---|---|
| Compound | Mean LC₅₀ | Mean LC₅₀ | Significance |
| NSC-302979 | 5.4 × 10⁻⁵ M* | 6.6 × 10⁻⁵ M** | Indistinguishable |
| | | | (P >> 0.05) |
| Curcumin | 6.1 × 10⁻⁵ M* | 6.8 × 10⁻⁵ M** | Indistinguishable |
| | | | (P >> 0.05) |
| NSC-156236 | Inactive | Inactive | — |

*results from 18 cell lines with Wild type p53
**results from 41-42 cell lines with mutant/deleted p53

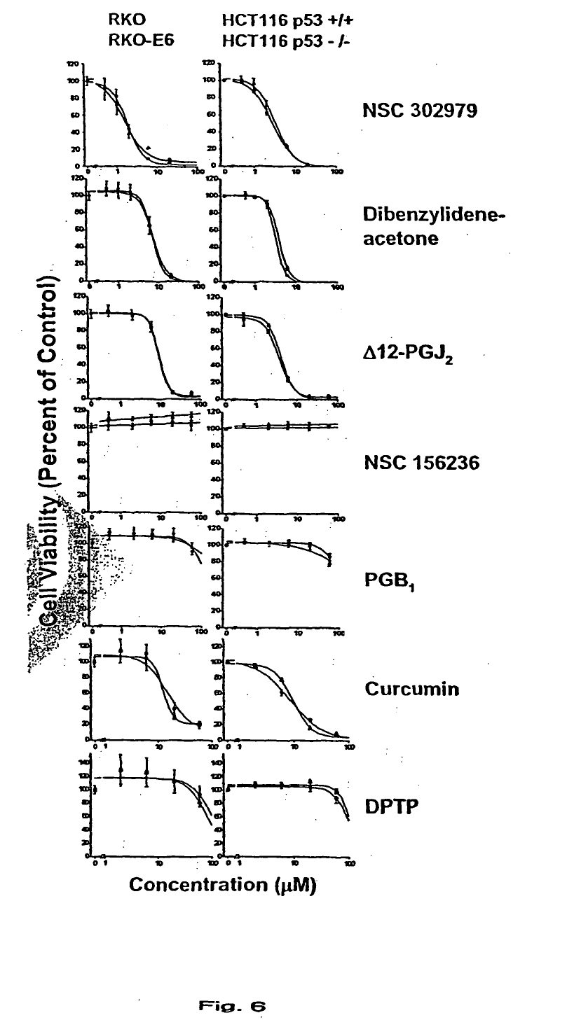

It is confirmed that isopeptidase inhibitors caused cell death independently of p53 by investigating their effects on two pairs of isogenic colon cancer cell lines. Isogenic HCT 116 +/+ and HCT 116 −/− cell lines have varying degrees of p53 haplosufficiency, p53 +/+ and p53 −/−, respectively (Bunz et al., 1999). NSC-302979, DBA, Δ12PGJ₂, and curcumin each caused cell death with equal potency (concentration for ½ maximal effect) and efficacy (maximal effect) in HCT 116 +/+ cells that are homozygous for p53 and HCT 116 −/− cells that are null for p53 [FIG. 6, right hand panels]. NSC-156236, PGB₁, and DPTP, which did not inhibit ubiquitin isopeptidase activity at concentrations <60 μM did not cause significant cell death in HCT 116 +/+ or HCT 116 −/− cells. Isogenic RKO and RKO-E6 cells accumulate p53 to varying degrees after genomic stress, due to the enhancement of p53 ubiquitination by HPV-E6. NSC-302979, DBA, Δ12PGJ₂, and curcumin each caused cell death with equal potency and efficacy in RKO and RKO-E6 cells [FIG. 6, left hand panels]. NSC-156236 PGB₁, and DPTP did not cause significant cell death in RKO or RKO-E6 cells.

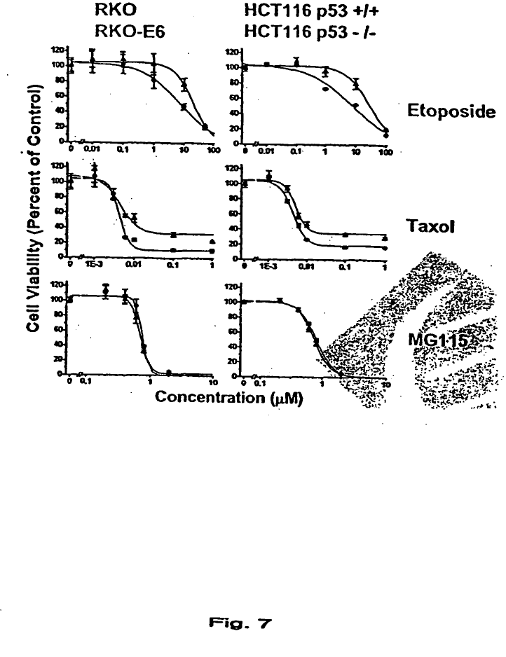

As procedural controls and for calibration etoposide and paclitaxel were evaluated on these same pairs of cells. Etoposide typifies agents that cause cell death via a p53-dependent pathway (Lowe et al., 1993). Accordingly, its potency in HCT 116 −/− cells was ˜4-fold less than its potency in HCT 116 +/+ cells [FIG. 7, top right panel]. Similarly, etoposide potency in RKO-E6 cells was ˜3-fold less than its potency in RKO cells [FIG. 7, top left panel]. Paclitaxel typifies an agent that causes cell death via a p53-independent pathway (O'Conner et al., 1997). Although its efficacy (maximal effect observed) in the HCT 116 −/− and RKO-E6 cells with dysfunctional p53 exceeded its efficacy in the corresponding HCT 116 +/+ and RKO cells, the potency of paclitaxel (concentration for ½ maximal effect) was equivalent in HCT 116 −/− compared to HCT 116 +/+ cells, as well as in RKO-E6 compared to RKO cells [FIG. 7, middle panel]. Lastly, MG115, an inhibitor of the 20S catalytic subunit of the proteasome was evaluated, to compare its effects with isopeptidase inhibitors. There are conflicting reports on the role of p53 in cell death caused by proteasome inhibitors like MG115 (Adams et al., 1999; Wagenknecht et al., 1999; An et al., 2000; Lopes et al., 1997; Shinohara et al., 1996; Dietrich et al., 1996). MG115 caused cell death with equal potency and efficacy in HCT 116 +/+ cells versus HCT 116 −/− and in RKO-E6 cells versus RKO cells, analogous to isopeptidase inhibitors [FIG. 7, bottom panel]. Table 2 summarizes the potency of all compounds as cell death agonists in both pairs of cell lines.

TABLE 2
Cytotoxic Potency (EC₅₀, μM) of Pharmacophore Panel and
Calibration Compounds in HCT 116 and RKO Cells with Different
p53 Status

| Compound | RKO | RKO-E6 | HCT^{+/+} | HCT^{-/-} |
|---|---|---|---|---|
| NSC-302979 | 1.8 ± 0.1 | 1.4 ± 0.1 | 2.7 ± 0.2 | 3.2 ± 0.2 |
| DBA | 7.0 ± 0.6 | 6.5 ± 1.1 | 3.2 ± 0.1 | 3.8 ± 0.1 |
| Δ12-PGJ₂ | 9.0 ± 0.8 | 9.2 ± 1.0 | 3.6 ± 0.3 | 4.1 ± 0.1 |
| Curcumin | 12.5 ± 5.9 | 14.9 ± 8.5 | 9.7 ± 1.1 | 8.6 ± 1.3 |
| NSC-156236 | >60 | >60 | >60 | >60 |
| PGB₁ | >60 | >60 | >60 | >60 |
| DPTP | >60 | >60 | >60 | >60 |
| Etoposide | 7.3 ± 1.6 | 20.0 ± 2.7 | 7.8 ± 3.9 | 29.0 ± 6.8 |
| Taxol | 0.004 ± 0.0 | 0.004 ± 0.0 | 0.004 ± 0.0 | 0.005 ± 0.0 |
| MG115 | 0.8 ± 0.0 | 0.7 ± 0.0 | 0.8 ± 0.0 | 0.7 ± 0.0 |

Accordingly, non-prostanoid classes of compounds, with α,β-unsaturated ketones, and one or two sterically accessible β-carbons will inhibit ubiquitin isopeptidase activity. It also been demonstrated that these compounds cause cell death independently of p53 tumor suppressor function in vitro. Specifically, the diterpene, NSC302979,the synthetic compound, DBA, the prostaglandin, Δ12-PGJ₂, and the curcuminoid, curcumin, all cause cell death with efficacy and potency that is indistinguishable (p>0.05) between HCT 116 p53 +/+ and HCT 116 p53 −/−, or RKO and RKO-E6 cells. Furthermore, cell death correlated with inhibition of isopeptidase activity. Regression analysis (IC₅₀ for inhibition of z-LRGG-AMC hydrolysis by isopeptidase versus IC₅₀ for cytotoxicity) yields a straight line with a correlation coefficient r²=0.93, n=7. Regression analysis (IC₅₀ for inhibition of ubiquitin-PEST hydrolysis by isopeptidase versus IC₅₀ for cytotoxicity) also yields a straight line with r²=0.73, n=13.

Inhibition of ubiquitin isopeptidase activity may propagate cell death by shifting the polyubiquitin chain length equilibrium to one of greater molecular weight. As a consequence of unfettered polyubiquitin chain growth, the pool of monoubiquitin diminishes. Alteration of monoubiquitin/polyubiquitin dynamics inevitably affects several transcription factors, other than p53 (Desterro et al., 2000). Furthermore, with depleted monoubiquitin pools, cells are hampered in their efforts to rid themselves of damaged/toxic proteins, eventually affecting protein-protein or protein-DNA interactions that modulate cell survival and apoptosis. Without being bound by any theory, the data appear to support a covalent mechanism, where α,β-unsaturated dienones covalently inhibit isopeptidases, possibly via their electrophilic β-carbons, e.g. Michael (1,4 addition) adduct formation between an isopeptidase cysteine residue and the β-carbon of a dienone. Compounds with sterically inaccessible or inert β-carbons (NSC-156236, PGB₁, and DPTP) were inactive as isopeptidase inhibitors.

The sub-structure search capabilities of the NCI DTP database (60 cell line screen) were used to identify NSC-302979 and NSC-156236, compounds used to test the pharmacophore and mechanism of action hypotheses. The results, along with the results by NCI scientists (Weinstein et al, 1997; Shi et al., 1998; O'Conner et al., 1997), exemplify the potential of this database and compound repository and the foresight of the NCI Developmental Therapeutics Branch. Others have suggested that the database content is misaligned with the goal to discover new anticancer drugs, based on a poor correlation between clonogenic survival and the NCI archival anti-proliferative activity (Brown, 1997). However, direct extension of data acquired in vitro to clinical situations in vivo is rarely straightforward. Used prudently, to enable or to advance mechanistic and pharmacophore hypotheses, the database supports the quest for anticancer drugs with novel structures and mechanisms of action.

The mechanistic and pharmacophore hypotheses are compatible with the structure-activity relationships reported by Kato et al., 1986; Sasaki et al., 1991; Sasaki and Fukushima, 1994. Kato et al. reported that Δ12-PGJ2 and several related Δ12PGJ₂ derivatives (all of which are cross-conjugated dienones) increased the life span of Ehrlich ascites tumor-bearing mice: i.p. doses of 20-30 mg/kg/day for five consecutive days prolonged survival 66-111%. In addition, Δ12PGJ₂ exhibits little cross-resistance with cisplatin and adriamycin, in vivo [Sasaki et al., 1991; Sasaki and Fukushima, 1994]. Despite these promising results, Δ12PGJ₂ is rapidly metabolized to an inactive compound (tin_{1/2}<5 min) in serum (Suzuki et al., 1998). Therefore, the discovery of isopeptidase inhibitors among chemical classes other than PG might be advantageous in surmounting any difficulties intrinsic to the antineoplastic development of the PG class.

It is unclear whether agents that inhibit the proteasome pathway cause cell death via a p53-independent process [Adams et al., 1999; Wagenknecht et al., 1999; An et al., 2000; Lopes et al., 1997; Shinohara et al., 1996; Dietrich et al., 1996]. The results here, with inhibitors of ubiquitin isopeptidase activity, and with a representative catalytic subunit inhibitor of the 20 S proteasome, support the conclusion that proteasome inhibition causes apoptosis independently of p53. This debate may originate from faulty assumptions about the competence of p53 that accumulates in cells treated with proteasome pathway inhibitors. For instance, genetically wild type p53 accumulates in the presence of the isopeptidase inhibitor Δ12PGJ₂, but in a conformationally and functionally impaired state (Moos et al., 2000; Mullally et al., 2001). An et al., 2000 have also reported that accumulation of wild type p53 protein and induction of apoptosis occur as independent markers of proteasome inhibition. Therefore one must use caution when interpreting the consequences of p53 accumulation without first testing its functionality.

The response to chemotherapy is complex and focus on a single factor, no matter how prominent, may exaggerate its role. However, numerous investigations show that disruption of p53 impairs the potency and efficacy of drugs used in oncology, e.g. 5-fluorouracil (O'Conner et al., 1997; Bunz et al., 1999; Lowe et al., 1994; Lowe, 1995; Weller, 1998; Pich, 1998; Mueller and Eppenberger, 1996; Karpf et al., 2001]. It is notable that vinca alkaloids, one of the few drug classes that act independently of p53 (O'Conner et al, 1997; Fan et al., 1998), may target the proteasome in addition to tubulin (Piccinini et al., 2001). LDP-341, the first proteasome inhibitor to enter clinical trials, appears to have a favorable safety and efficacy profile (Dalton et al., 2001). Clinical studies to evaluate proteasome inhibition as an adjuvant to systemic chemotherapy are also currently in development (Cusack et al., 2001). The results here demonstrate that another component of the proteasome pathway, isopeptidase activity, at least warrants further investigation as a target for antineoplastic drug discovery.

Demonstration that Punaglandins are Potent Inhibitors of Ubiquitin Isopeptidase

Isolation of Punaglandins. The octocoral, Telesto riisei was collected in Hawaii, August 2001. The freeze-dried organism was macerated and subjected to a hexane-soxhlet extraction (Baker et al., J. Nat. Prod., 57, 1346-1353, 1994). The hexane-soluble material was partitioned against 70% CH₃OH/30% H₂O resulting in hexane and aqueous CH₃OH fractions. ¹H NMR confirmed punaglandins in the aqueous fraction. The aqueous-soluble material was subjected to silica-gel flash chromatography yielding three fractions containing mixtures of punaglandins as determined by ¹H NMR. Each fraction was further purified by reversed-phase C-18 HPLC leading to the following punaglandins: PNG 2 (6), PNG 3 (7), PNG 4 (8), Z-PNG 4 (9), and PNG 6 (10).

Structure Determination of Punaglandins& The structures of all isolated punaglandins were confirmed by ¹H and ¹³C NMR, and EIMS. Additionally, HMQC, HMBC, and DEPT data were collected for PNG 2 (6), Z-PNG 4 (9), and PNG 6 (10). All data were consistent with literature values (Baker et al., J. Nat. Prod., 57, 1346-1353, 1994). ¹H and ¹³C NMR, and EIMS data are provided in the experimental section.

Biological Results:

Isopeptidase Assay. It was hypothesized that the punaglandins would inhibit ubiquitin-isopeptidase activity of the proteasome pathway as does the prostaglandin, Δ¹²-PGJ₂ (1), but with greater potency because of the chlorinated C-10. To validate this theory, cellular isopeptidase enzymatic activity was measured in vitro after treatment with individual punaglandins, PNG 2 (6), PNG 3 (7), and Z-PNG 4 (9) and in vivo after individual treatment with PNG 4 (8) and PNG 6 (10). This activity was determined utilizing the simple isopeptidase substrate, ubiquitin-PEST (Ub-PEST), a full/length ubiquitin (Ub) molecule fused with an 18-amino acid C-terminal peptide extension that most isopeptidases use as a substrate. The polypeptide extension contains regions rich in Pro (P), Glu (E), Ser (S), and Thr (T) residues called PEST motifs that are responsible for rapid degradation of these and other unstable proteins. Ub isopeptidases specifically cleave this peptide extension, yielding free, full-length Ub. Both the fused Ub-PEST (10.5 kDa) and the cleaved, free Ub (8.5 kDa) can be observed via immunochemical detection.

In Vitro Isopeptidase Activity. PNG 2 (6), PNG 3 (7), and Z-PNG 4 (9) were initially analyzed for isopeptidase activity in vitro in HCT 116 lysates. Cells were lysed with isopeptidase activity buffer and protein concentration adjusted to 0.3 mg/mL per sample. Each sample was subsequently incubated with 2 μM, 6 μM, 20 μM, 60 μM, or 200 μM of PNG 2 (6), PNG 3 (7), Z-PNG 4 (9), Δ¹²-PGJ₂ (1), and PGA₁ (2) in addition to 50 μg/ml of Ub-PEST. Negative control cells were treated with vehicle (0.5% DMSO) and equivalent Ub-PEST. The reaction was terminated after 45 min, a timepoint which was previously determined to demonstrate Ub-PEST degradation (data not shown). The amount of fused Ub-PEST and free Ub product was determined by SDS-PAGE fractionation.

The results obtained were as anticipated. The vehicle control cells revealed a 1:1 ratio of fused Ub-PEST (10.5 kDa) to free Ub (8.5 kDa) as predicted to occur at a 45 min time interval. The simple dienone PGA₁ (2) showed no detectable inhibition of Ub-PEST and was comparable to vehicle treated lysates, indicating no inhibition of isopeptidase activity. Δ¹²-PGJ₂ (1) inhibited Ub-PEST degradation initially at 60 μM with apparent total inhibition at 200 μM. These results are in agreement with previously reported data for 1 (Mullally et al., J. Biol. Chem, 276, 30366-30373, 2001). As hypothesized, the punaglandins inhibited Ub-PEST degradation, preventing free Ub formation. Inhibition was initially observed at 20 μM with complete inhibition occurring at 60 μM indicating that PNG 2 (6), PNG 3 (7), Z-PNG 4 (9) are more potent inhibitors of isopeptidase activity in vitro than Δ¹²-PGJ₂ (1).

In vivo Isopeptidase Activity. To investigate whether punaglandins can inhibit isopeptidase activity in vivo, isopeptidase activity was analyzed after punaglandin treatment of RKO cells. PNG 4 (8) and PNG 6 (10) were selected for in vivo investigation because they differ only in enone functionality, where (8) is a dienone and (10) an enone. RKO cells were incubated with 1 μM, 4 μM, or 7 μM of PNG 4 (8) and PNG 6 (10). As a negative control, cells were incubated with vehicle (0.5% DMSO) or the protein synthesis inlnbitor cycloheximide (CHI), which does not inhibit isopeptidase activity. As a positive control, cells were treated with a known isopeptidase inhibitor, shikoccin (SK). After 6 h drug treatment, cells were lysed and protein concentration adjusted to 0.3 mg/mL per sample. Each sample was subsequently incubated with 50 μg/mL of Ub-PEST. The reaction was terminated after 20 min and the respective amounts of Ub-PEST (10 kDa) and free Ub product (8.5 kDa) were determined by SDS-PAGE fractionation.

The in vivo data confirmed isopeptidase inhibition by PNG 4 (8) and PNG 6 (10). Control cells treated with vehicle or cycloheximide produced a 1:1 ratio of Ub-PEST to free Ub similar to no treatment (data not shown). The positive control, shikoccin showed expected inhibition of Ub-PEST degradation as did 8 and 10 with noticeable inhibition occurring at 7 μM. Previous in vivo studies of Δ¹²-PGJ₂ (1) in cells treated for 12 h showed inhibition of Ub-PEST degradation at 60 μM (Mullally et al., Medicinal Chemistry, University of Utah: Salt Lake City,. 2003). This in vivo data corresponds well with in vitro data suggesting that PNG 4 (8) and PNG 6 (10) inhibit isopeptidase activity in vivo more potently than Δ¹²-PGJ₂ (1). Densitometry measurements verified that Ub-Pest degradation at 7 μM for 8 is 67% of the control compared to 75% for 10.

p53. Dependence. Proteasome inhibitors cause rapid accumulation of substrates normally degraded by the ubiquitin proteasome pathway. For instance, p53 and p53-inducible gene products such as p21 are regulated by this pathway. The ubiquitin proteasome pathway post-transcriptionally regulates the amount of cellular p53 via murine double minute clone (MDM2) that directly interacts with p53 and promotes its ubiquitination and proteasomal degradation (Haupt et al., Nature, 387, 296-299, 1997). p53 is typically latent until activated by cellular stress or DNA damage, which causes disruption of the p53-MDM2 complex. Once dissociated, p53 accumulates and binds DNA, initiating transcription of genes that trigger growth arrest, DNA repair, and apoptosis.

Characteristic of proteasome pathway inhibitors, treatment of cells with Δ¹²-PGJ₂ (1) inhibits degradation of the tumor-suppressor protein p53 causing it to accumulate in cells. Since punaglandins also inhibit ubiquitin isopeptidase activity, we hypothesized that PNG 4 (8) and PNG 6 (10) also inhibit degradation of p53 by inhibiting its proteasomal degradation. To determine if our hypothesis was correct both PNGs were assayed in isogenic colon cancer cell lines RKO and RKO-E6. RKO-E6 cells are isogenic variants of RKO cells stably transfected with the HPV-E6 (human papilloma virus derived E6) oncoprotein. The E6 viral oncoprotein recruits and binds a cellular ubiquitin-protein ligase E6-AP (E6-associated protein)(Scheffner et al., Cell, 63, 1129-1136, 1990). This E6-AP complex specifically interacts with p53 targeting it for ubiquitin-proteasome-mediated degradation, thus increasing degradation of p53 (Scheffner et al., Cell, 75, 495-505, 1993; Ashcroft et al., Oncogene, 18, 7637-7643, 1999). Generally, p53 levels are significantly lower in RKO-E6 cells compared to RKO cells due to increased degradation of p53. When these cells are treated with ubiquitin-proteasome pathway inhibitors, p53 accumulates in RKO and RKO-E6 cells to a similar extent.

To determine if punaglandins inhibit p53 degradation by disrupting the proteasome pathway, the accumulation ratio of p53 was determined in RKO and RKO-E6 cells treated with selected punaglandins, PNG 4 (8) and PNG 6 (10). Equally seeded RKO and RKO-E6 cells were treated with vehicle (0.5% DMSO), 50 μM Etoposide, 60 μM PGA₂ (3), 7 μM PNG 6 (10), and 7 μM PNG 4 (8). After 6 h incubation with vehicle or drug, cells were lysed and harvester. Fifteen μg/mL of protein from each lysate was fractionated by SDS-PAGE and probed for p53 protein. The p53 band intensities were then analyzed and quantitated using densitometry. The ratio of p53 in RKO-E6 to RKO cells was calculated by dividing the amount of p53 in RKO-E6 cells by the amount in RKO cells and multiplying by 100 to give percent

Control RKO-E6 cells (vehicle treated) show less accumulated p53 protein than the control RKO (60%) as expected due to RKO-E6's specifically enhanced p53 degradation.

Also etoposide, a topoisomerase II inhibitor that induces p53 activation but does not inhibit the proteasome pathway, was examined in the RKO and RKO-E6 cell lines. Due to increased p53 degradation by E6, less p53 accumulated in RKO-E6 cells as compared to RKO cells (50%). PGA₂ (3), an enone PG that negligibly inhibits isopeptidase activity, shows results comparable to the control treatment with regard to p53 accumulation in RKO-E6 cells at 60% of RKO cells. As hypothesized, PNG 6 (10) and PNG 4 (8) assayed at a lower concentration of 7 μM, versus Δ¹²-PGJ₂ (1), assayed at 60 μM (data not shown), showed enhanced accumulation of p53 in the RKO and RKO-E6 cells to Δ¹²-PGJ₂ (1). This enhanced accumulation of the proteasomal regulated p53 protein in both cell lines is consistent with activity expected of ubiquitin-isopeptidase inhibitors. It is interesting to note that although the enone PNG 6 (10) induces significant p53 accumulation in both cell lines there appears to be slightly less accumulation in the RKO-E6 cell line.

p21 Accumulation. The dienone prostaglandin, Δ¹²-PGJ₂ (1), and the enone PGs, PGA₁ (2) and PGA₂ (3), have been shown to electrophilically alter wt p53 confirmation causing accumulation of transcriptionally silent p53 in the cytosol (Moos et al., Proc. Natl. Acad. Sci. USA, 97, 9215-9220, 2000; Mullally et al., J. Biol. Chem, 276, 30366-30373, 2001). This inactivation of p53 eliminates p53-dependent transactivation of p21^{Waf/cip-1} (p21)( Moos et al., Proc. Natl. Acad. Sci. USA, 97, 9215-9220, 2000). RKO and HCT 116 cells treated with PGA₁ (2) and PGA₂ (3) show an increase in mutant p53 protein levels and a decrease in p21 mRNA transcription and p21 protein induction. Unlike the enones, 2 and 3, the dienone Δ¹²-PGJ₂ (1) causes a decline in p21 mRNA but an increase in accumulated p21 protein. This differential activity is attributed to Δ¹²-PGJ₂'s ability to preferentially inhibit ubiquitin-isopeptidase activity via its cross-conjugated α,β-unsaturated ketone, thus decreasing transcription and degradation of p21. As expected PGB₁ (4), containing a sterically hindered and therefore less active α,β-unsaturated ketone does not alter p53 protein conformation, expression, or transactivation.

It was therefore hypothesized that the isopeptidase inhibitors PNG 4 (8) and PNG 6 (10) would cause a similar accumulation of p21 protein. RKO cells were treated with 2 μM and 7 μM of 8 and 10, 50 μM Etoposide, 60 μM PGA₂ (3), or 0.5% DMSO for 6 h before harvesting. Protein was then quantitated, fractionated by SDS-PAGE, and probed for p21 protein. The p21 band intensities were then analyzed and quantitated using densitometry. The ratio of p21 in treated cells to control cells was calculated by dividing the amount of p21 in treated cells by the amount in control cells and multiplied by 100 to give percent.

DMSO treated controls exhibit negligible accumulation of p21 protein and etoposide shows significant induction of p21, 290% over control. Also treatment with the enone PGA₂ (3) almost completely abrogates p21, to 10% of the control. The punaglandins show p21 accumulation, 90-290% of control. Notably, the enone punaglandin, PNG 6 (10) shows significantly less p21 accumulation than the dienone PNG 4 (8).

Caspase Assay. Another characteristic function of isopeptidase inhibitors is their ability to cause apoptosis independently of p53 (Mullally et al., J. Biol. Chem, 276, 30366-30373, 2001; Mullally et al., Mol. Pharmacol, 62, 2002). Many types of mammalian cells undergo apoptosis during normal development or in response to a variety of stimuli. The ability to induce apoptosis is constructive for any anti-neoplastic agent. The involvement of the ubiquitin-proteasome in apoptosis has been established yet its precise role remains unresolved. It is most likely linked to its ability to degrade cellular proteins that are intimately involved in apoptosis regulation. In most cell lines proteasome inhibitors have been shown to trigger apoptosis upstream of the caspase cascade (Orlowski et al., Cell Death Differ., 6, 303-313, 1999). Although both the dienone Δ¹²-PGJ₂ (1) and enone PGs, PGA₁ (2) and PGA₂ (3) have been shown to alter p53 protein conformation in RKO and HCT 116 cells, only the isopeptidase-inhibitor prostaglandin, Δ¹²-PGJ₂ (1) potently causes apoptosis independently of p53 (Mullally et al, J. Biol. Chem, 276, 30366-30373, 2001; Moos et al., Proc. Natl. Acad. Sci. USA, 97, 9215-9220,2000).

The J series prostaglandins have been shown to induce apoptosis in a time dependent manner in RKO cells Mullally et al., J. Biol. Chem, 276, 30366-30373, 2001). To determine if selected punaglandins, PNG 4 (8) and PNG 6 (10) also induce apoptosis in RKO cells in a time dependent manner, caspase-3 activity was determined. During apoptosis the caspases, a family of cysteinyl aspartate-specific proteases, are activated. These enzymes cleave specific substrates resulting in biochemical and morphological changes associated with apoptosis. Therefore, detection of activated caspases can be used as a biochemical marker for apoptosis. Caspase-3 has substrate specificity for the amino acid sequence Asp-Glu-Val-Asp (DEVD), cleaving after Asp, and therefore caspase activity can be measured by monitoring cleavage of the substrate DEVD-AMC (Asp-Glu-Val-Asp-7-amino-4-aminomethylcoumarin).

RKO cells were treated for 4, 8, and 12 h with PNG 4 (8) at 0.5 μM and PNG 6 (10) at 1 μM. Taxol, a known apoptotic inducer at 1 μM, was used as a positive control, and negative control cells were treated with vehicle (0.5% DMSO). Caspase-3 activity rose steadily as expected from 4-12 h in cells treated with PNG 4 (8), PNG 6 (10), and Taxol but remained constant in the untreated control cells. These results confirm that both the dienone PNG 4 (8) and the enone PNG 6 (10) cause apoptosis in RKO cells as expected.

Cytotoxicity in Human Colon Tumor (HCT) Cell Lines. To determine if punaglandins elicit cytotoxicity independently of the tumor-suppressor p53, as do other dienone prostaglandins, several cytotoxicity assays were employed. First, cell death effects of all isolated punaglandins were investigated in pairs of isogenic colon cancer cell lines, the HCT 116^{+/+} competent cell line and HCT 116^{-/-} null cell lines. HCT 116^{+/+} cells are homozygous for p53^{+/+} and p21^{+/+}; where as, HCT 116^{-/-} cells lack both alleles for p53 (p53^{-/-}) or p21 (p21^{-/-}).

Each cell line was seeded simultaneously in 96-well plates and incubated for 24 h before treatment with individual punaglandins at various concentrations for 48 additional h. Cytotoxicity was determined using MTS and absorbances were recorded at 490 nm. Half maximal effective concentrations (EC₅₀) and Log EC₅₀s were calculated from a concentration-response curve generated using GraphPad Prism (Table 1).

TABLE 1
Cytotoxicity of isolated punaglandins in HCT 116 (p53 wt),
(p53^{-/-}), (p21 wt), and (p21^{-/-}) cell lines.

| | Log EC₅₀ +/− Standard Error of Log EC₅₀ | | | |
|---|---|---|---|---|
| | HCT 116 | HCT 116 | HCT 116 | HCT 116 |
| cmpd | (p53 wt) μM | (p53^{-/-}) μM | (p21 wt) μM | (p21^{-/-}) μM |
| 6 | −1.40 ± 0.06 | −1.40 ± 0.05 | −1.33 ± 0.05 | −1.40 ± 0.05 |
| 7 | −0.49 ± 0.09 | −0.43 ± 0.09 | −0.54 ± 0.07 | −0.53 ± 0.08 |
| 8 | −0.48 ± 0.09 | −0.45 ± 0.40 | −0.47 ± 0.09 | −0.55 ± 0.08 |
| 9 | −1.50 ± 0.09 | −1.57 ± 0.15 | −1.53 ± 0.09 | −1.49 ± 0.08 |
| 10 | −0.47 ± 0.08 | −0.44 ± 0.08 | −0.48 ± 0.09 | −0.49 ± 0.09 |

Each punaglandin caused cell death with equal potency and efficacy in HCT 116 p53^{+/+} and p21^{+/+} as compared to the corresponding HCT 116 p53^{-/-} and p21^{-/-} null cell lines, respectively (Table 1). These results are indicative of a p53-independent mechanism. Punaglandins are also more potent than PGA₁ (2) and Δ¹²-PGJ₂ (1) in competent HCT 116 cells, which display EC₅₀s of 11.6 μM and 3.6 μM, respectively (Mullally et al., Medicinal Chemistry, University of Utah: Salt Lake City, 2003). Interestingly, PNG 2 (6) and Z-PNG 4 (9) have increased cytotoxicity (EC₅₀=0.04 and 0.03 μM, respectively) compared to PNG 3 (7), PNG 4 (8), and PNG 6 (10) (average EC₅₀˜0.35). This result may be due to different spatial orientations of PNG 2 (6) and Z-PNG 4 (9) that exhibit less steric hindrance toward nucleophilic addition. Suzuki et al. suggested that a 1,3-interaction between the C(8) and C(11) substituents on PGA₁ (2), may control stereoselectivity of thiol addition at C(11) (Suzuki et al., J. Am. Chem. Soc., 119, 2376-2385, 1997). Perhaps a similar steric interaction exists in (7), (8), and (10) thus slightly decreasing cytotoxicity.

Cytotoxicity in Human Colon Tumor (RKO) Cell Lines. Punaglandins (8) and (10) were also assayed for p53-independent cytotoxicity in RKO and RKO-E6 cells. Each cell line was seeded simultaneously in 96-well plates and incubated for 24 h before treatment with PNG 4 (8) and PNG 6 (10) at various concentrations for 48 h. Cytotoxicity was determined using MTS and absorbances were recorded at 490 nm. Log EC₅₀s and EC50s were calculated from a concentration-response curve generated using GraphPad Prism (Table 2).

The punaglandins caused cell death with equal potency and efficacy in RKO cells (competent p53) and RKO-E6 (disrupted p53) cell lines. These data substantiate that the cytotoxicity mechanism of the punaglandins occurs independently of p53. Additionally, PNG 4 (8) and PNG 6 (10) are more potent in RKO cell lines than PGA₁ (2) and Δ¹²-PGJ₂ (1) generating EC_{50′}s of 33.6 μM and 9.0 μM, respectively versus 0.31 μM for 8 and 0.44 μM for 10.

TABLE 2
Cytotoxicity of tested PNG 4 and PNG 6 in RKO and RKO-E6
cell lines.

| | Log EC₅₀ +/− Standard Error | |
|---|---|---|
| | of Log EC₅₀ | |
| | RKO | RKO-E6 |
| cmpd | μM | μM |
| 8 | −0.51 ± 0.13 | −0.43 ± 0.14 |
| 10 | −0.36 ± 0.11 | −0.33 ± 0.11 |

### REFERENCES

Adams et al., Cancer Res 59:2615-2622 (1999).

An et al., Leukemia 14:1276-1283 (2000).

Ashcroft et al., Oncogene 18-7637-7643 (1999).

Bradford et al., Anal Biochem 72.248-254 (1976).

Brown et al., Oncol Res 9:213-215 (1997).

Bunz et al., J Clin. Invest. 104:263-269 (1999).

Cusack et al., Cancer Res 61:3535-3540 (2001).

Dalton et al., Am Soc Hematot Educ Program) January:157-177 (2001).

Dang et al., Biochemistry 37,1868-1879 (1998).

Desterm et al., Cell Mot Life Sci 57:1207-1219 (2000).

Dietrich et al., Proc. Natl. Aced Sci USA 93-10815-10819 (1996).

Fan et al., Clin Cancer Res 4:1047-1054 (1998).

Fenteany et al., Science (Wash D.C.) 268:726-731 (1995).

Hershko et al., Annu Rev Biochem 67:425-479 (1998).

Hochstrasser, Annu Rev Genet 30-405439 (1996).

Karpf et al., Mol Pharmacol 59-751-757 (2001).

Kato et al., Cancer Res 46:3538-3542 (1986).

Msselev et al., Chem BW 8:739-758 (2000).

Lopes et al., J Biol. Chem 272:12893-12896 (1997).

Lowe et al., Cell 74.957-967 (1993).

Uwe et al., Science (Wash D.C.) 266:807-810 (1994).

Uwe, Curr Opin Oncol 7:547-553 (1995).

Meng et al., Cancer Res 59-2798-2801 (1999).

Monks et al., Anticancer Drug Dev 12:533-541 (1997).

Moos et al., Proc. Natl Acad Sci USA 97:9215-9220 (2000).

Mueller et al., Anticancer Res 16.3845-3848 (1996).

Mullally et al., J Biol Chem 276:30366-30373 (2001).

O'Connor et al., Cancer Res 574285-4300(1997).

Piceinini et al., Biochem J 356:835-841 (2001).

Pich, The Cancer Journal 11-223-228 (1998).

Rodriguez et al., J Med Chem 40-1827-1834 (1997).

Sasaki et al., Anticancer Drugs 5:131-138 (1994).

Sasaki et al., Gynecol Oncol 41:36-40(1991).

Suzuki et al., J Med Chem 41:3084-3090 (1998).

Shi et al., Mol Pharmacol 53:241-251 (1998).

Shinohara et al., Biochem J 317:385-388 (1996).

Wagenknecht et al., Cell Physiol Biochem 9:117-125 (1999).

Weinstein et al., Science (Wash D.C.) 275:343-349 (1997).

Weller, Cell Tissue Res 292:435-445 (1998).

Yin et al., Biochemistry 39-10001-10010 (2001).

The contents of provisional application Ser. No. 60/396,584, filed Jul. 18, 2002, are hereby incorporated by reference in their entirety.

1. A method of inhibiting a ubiquitin isopeptidase in a cell, comprising contacting said cell with an effective amount of a composition comprising a compound having an α,β,-unsaturated ketone, wherein said ketone has a sterically accessible electrophilic β-carbon, wherein said agent is cell permeable and active in intact cells, and wherein said agent is not a cyclopentenane prostaglandin of the J family.

2. The method according to claim 1, wherein said compound contains a cross-conjugated α,β, α′,β′-unsaturated ketone moiety, and wherein at least one of said electrophilic β carbons is sterically accessible.

3. The method according to claim 2, wherein both of said electrophilic β carbons are sterically accessible.

4. The method according to claim 2, wherein the α carbon of at least one α,β-unsaturated ketone moiety bears an electron withdrawing substituent.

5. The method according to claim 4, wherein said electron withdrawing substituent is selected from the group consisting of fluorine, chlorine, bromine, iodine, nitro, nitrilo and carboxy.

6. The method according to claim 5, wherein said carboxy group is an acid, ester of amide group.

7. The method according to claim 1, wherein said α,β-unsaturated ketone comprises a conjugated cyclopentene moiety.

8. The method according to claim 1, wherein said compound is selected from the group consisting of dibenzylideneacetone (DBA), curcumin, shikoccin (NSC-302979), shikoccin epoxide, O-methyl shikoccin, O-methyl shikoccin epoxide, shikodomedin, rabdoshikoccin A, rabdoshikoccin B, rabdolatifolin, rabdoumbrasanin, and a punaglandin.

9. The method according to claim 8, wherein said compound is a punaglandin selected from the group consisting of PNG 2, PNG3, PNG4, Z-PNG4, and PNG 6.

10. A method of treating or alleviating an oncological malady in a subject, comprising administering to said subject a composition comprising an effective amount of a ubiquitin isopeptidase inhibitor.

11. The method according to claim 10, wherein said inhibitor comprises an α,β,-unsaturated ketone group having a sterically accessible electrophilic β-carbon, wherein said inhibitor is cell permeable and active in intact cells, and wherein said inhibitor is not a cyclopentenone prostaglandin of the J family.

12. The method according to claim 11, wherein said inhibitor contains a cross-conjugated α,β, α′,β′, -unsaturated ketone moiety, and wherein at least one of said electrophilic, β carbons is sterically accessible.

13. The method according to claim 12, wherein both of said electrophilic β carbons are sterically accessible.

14. The method according to claim 12, wherein the α carbon of at least one α,β-unsaturated ketone moiety bears an electron withdrawing substituent.

15. The method according to claim 14, wherein said electron withdrawing substituent is selected from the group consisting of fluorine, chlorine, bromine, iodine, nitro, nitrilo and carboxy groups.

16. The method according to claim 15, wherein said carboxy group is an acid, ester of amide group.

17. The method according to claim 11, wherein said α,β-unsaturated ketone comprises a conjugated cyclopentene moiety.

18. The method according to claim 11, wherein said compound is selected from the group consisting of dibenzylideneacetone (DBA), curcumin, shikoccin (NSC-302979), shikoccin epoxide, O-methyl shikoccin, O-methyl shikoccin epoxide, shikodomedin, rabdoshikoccin A, rabdoshikoccin B, rabdolatifolin, rabdoumbrasanin, and a punaglandin.

19. The method according to claim 18, wherein said compound is a punaglandin selected from the group consisting of PNG 2, PNG3, PNG4, Z-PNG4, and PNG 6.

20. The method according to claim 1 wherein said cell is a human cell.

21. The method according to claim 10, wherein said subject is a human.

22. The method according to claim 10, wherein said oncological malady is selected from the group consisting of tumors of the head and neck, esophagus, stomach, ileum, colon, rectum, breast, ovary, prostate, testes, lung, brain, kidney, liver, pancrease, muscle (sarcoma), connective tissue (sarcoma) or fat (sarcoma), bone marrow, lymphomas and leukemias.

23. A pharmaceutical composition suitable for treating an oncological malady in a human subject, comprising an effective amount of a ubiquitin isopeptidase inhibitor, wherein said inhibitor is not a cyclopentenone prostaglandin of the J family.

24. The composition according to claim 23, wherein said inhibitor comprises an α,β-unsaturated ketone moiety, wherein said ketone has a sterically accessible electrophilic β-carbon, wherein said agent is cell permeable and active in intact cells, and wherein said agent is not a cyclopentenone prostaglandin of the J family, together with a pharmaceutically acceptable carrier, excipient, or diluent.

25. The method according to claim 10, wherein said composition further comprises an effective amount of at least one additional pharmaceutically active antineoplastic agent.

26. The method according to claim 25, wherein said additional antineoplastic agent is selected from the group consisting of a topoisomerase 2 inhibitor, a DNA methyltransferase inhibitor, a topoisomerase 1 inhibitor, and a cyclopentenone prostaglandin of the J series.

27. The method according to claim 26, wherein said additional agent is selected from the group consisting of etoposide, decitibine, and an active camptothecin analog.

28-35. (canceled)

36. A method of treating a disorder mediated by a proteosome pathway in a patient, comprising administering to said patient an effective amount of a ubiquitin isopeptidase inhibitor, wherein said disorder is selected from the group consisting of, dry eye disorder, restenosis, inflammation, autoimmune disorder, graft rejection, ischemia, cachexia, muscle wasting and loss of bone or hair.

37. The method according to claim 36, wherein the disorder is dry eye disorder.

38. The method according to claim 36, wherein the disorder is restenosis.

39. The method according to claim 36, wherein the disorder is inflammation.

40. The method according to claim 36, wherein the disorder is an autoimmune disorder.

41. The method according to claim 36, wherein the disorder is graft rejection.

42. The method according to claim 36, wherein the disorder is ischemia.

43. The method according to claim 36, wherein the disorder is cachexia.

44. The method according to claim 36, wherein the disorder is muscle wasting.

45. The method according to claim 36, wherein the disorder is a loss of bone or hair.
